(19) United States
(12) Patent Application Publication
(10) Pub. No.: US 2026/0272820 A1
(43) Pub. Date: Sep. 17, 2026

(54) SKIN ARCHAEA PREPARATION

(71) Applicant: Universität Wien, Wien (AT)

(72) Inventors: Christa Schleper, Wien (AT); Michael Melcher, Wien (AT); Ülkü Perier, Wien (AT); Logan Hodgskiss, Wien (AT); Melina Kerou, Wien (AT); Christine Moissl-Eichinger, Graz (AT); Alexander Mahnert, Graz (AT)

(73) Assignee: Universität Wien, Wien (AT)

(21) Appl. No.: 19/165,132

(22) PCT Filed: Mar. 13, 2024
(86) PCT No.: PCT/EP2024/056658

§ 371 Date: Sep. 14, 2025

(30) Foreign Application Priority Data
Mar. 13, 2023 (EP) 23161567.5

(51) Int. Cl. A61K 8/99; A61K 35/66; A61Q 5/00; A61Q 19/00; A61Q 19/08

(52) U.S. Cl. CPC A61K 8/99; A61K 35/66; A61Q 5/00; A61Q 19/00; A61Q 19/004; A61Q 19/005; A61Q 19/007; A61Q 19/08; A61K 2800/10

(57) ABSTRACT

A preparation comprising skin-specific ammonia-oxidizing archaea (sAOA) of the phylum Thermoproteota and genus Nitrosocosmicus, which comprise at least one of the ssp strains R2S, T1S, X2B, or Z3A, which are each characterized by a unique amoA sequence.

### FIELD OF THE INVENTION

The invention refers to preparations comprising ammonia-oxidizing archaea (AOA) of the phylum Thermoproteota which AOA are of one or more strains originating from human skin (sAOA), or probiotic fractions thereof, cosmetic and pharmaceutical compositions comprising such preparations, and their use in treating human beings. The invention further relates to methods of producing said sAOA preparations.

### BACKGROUND OF THE INVENTION

Most microorganisms in the environment are beneficial and perform important roles in mineralizing nutrients, producing essential vitamins or—when associated with plants and animals-serving as protective barriers and activating the immune system. The same is true for the complex microbiota associated with the human body, the so-called microbiome. On human skin, microorganisms serve primarily as an important barrier against pathogens, in particular by maintaining skin pH, inducing anti-inflammatory immune responses and promoting skin healing [1,2]. They are also essential in the maturation process of cutaneous immunity, effectively “training” the cutaneous immune system [3,4]. Inflammatory and allergic diseases of the skin, such as atopic dermatitis, acne, psoriasis and dandruff are often accompanied by an imbalance (dysbiosis) of the skin microbiome [5-8], exacerbated by the increased availability and application of “harsh” cosmetic products [9, 10]. Therefore solutions are needed to treat particularly chronic skin conditions and diseases by trying to reconstitute a healthy skin microbiome (e.g [11, 12]) similar to the use of probiotics to improve gut health.

The Human Microbiome Project, founded to decipher the entire set of microorganisms associated with the human body, continues to provide valuable information on how microbial diversity correlates with the health status of humans.

Only recently it has been recognized that not only bacteria and fungi, but also archaea are part of the human skin microbiome [13-19]. Based on 16S rRNA gene copies Archaea comprised up to 4.2% of the prokaryotic skin microbiome. Most of the gene signatures analyzed belonged to ammonia-oxidizing archaea of the phylum Thermoproteota, a group of Archaea which was also found in hospitals and clean room facilities. The metabolic potential for ammonia oxidation of the skin-associated Archaea was supported by the successful detection of archaeal amoA genes in human skin samples [13].

Moissl-Eichinger et al. describe amplicon sequencing of skin samples to reveal the prevalence of specific euryarchaeal and mainly AOA taxa, represented by a core archaeome of the human skin.

The ammonia oxidizing archaea of the phylum Thermoproteota, (formerly known as Thaumarchaeota) was reviewed by Stieglmeier et al. (The Prokaryotes—Other Major Lineages of Bacteria and the Archaea; E. Rosenberg et al. (eds.), DOI 10.1007/978-3-642-38954-2_338, Springer-Verlag Berlin Heidelberg 2014). AmoA genes are considered a distinctive feature for this phylum. However, the lack of cultured representatives and further genomic information regarding these organisms has left their taxonomic assignment ambiguous.

U.S. Pat. No. 7,820,420 discloses compositions including ammonia-oxidizing bacteria to increase production of nitric oxide and nitric oxide precursors and methods of using the same. Ammonia-oxidizing bacteria are selected from the group consisting of any of Nitrosomonas, Nitrosococcus, Nitrosospira, Nitrosocystis, Nitrosolobus, Nitrosovibrio, and combinations thereof.

WO2019104135 discloses topical use of ammonia oxidizing microorganisms, disclosing ammonia-oxidizing bacteria and ammonia-oxidizing archaea.

Nicol G W et al. describe the genome sequence of “Candidatus Nitrosocosmicus franklandus” C13, a terrestrial ammonia-oxidizing archaeon, which is isolated from soil.

There is a need for new probiotics to treat human skin, particularly to improve the human skin microbiome, which could act as an important barrier protecting the human body from pathogens and other environmental influences.

### SUMMARY OF THE INVENTION

It is the objective of the invention to provide a probiotic preparation for modulating the microbiota of the human body. It is a particular objective to provide suitable preparations of skin-resident microbes with a high potential to improve skin treatment.

The objective is solved by the subject matter as claimed and as further described herein.

The invention provides for new preparations of ammonia-oxidizing archaea (AOA) which are skin-specific. New skin-specific AOA (sAOA) were isolated from human skin, and were found to be different from known AOA of environmental sources other than skin. The sAOA were isolated from healthy human skin and have the potential to improve skin treatments. SAOA not only have the potential for cosmetic purposes of treating skin, but also to act in a therapeutic manner, such as to maintain the homeostasis of the nitrogen cycle in the skin environment.

The sAOA are understood to be specific insofar as being adapted to diverse human sub-populations, and it is perceived that the risk of potential incompatibility or adverse effects on humans is mitigated, which enables the production of archaeal preparations beneficial to skin.

The invention provides for a preparation comprising skin-specific ammonia-oxidizing archaea (sAOA) of the phylum Thermoproteota and genus Nitrosocosmicus, which comprise at least one of the ssp strains R2S, T1S, X2B, or Z3A.

Specifically, the strain T1S comprises a genomic sequence SEQ ID NO:19. Strain T1S has been identified as Candidatus N. epidermidis.

Specifically, the strain R2S comprises a genomic sequence SEQ ID NO:20. Strain R2S has been identifed as Candidatus N. unguis. Subspecies (ssp) strains may comprise variant genomic sequences with a certain sequence similarity to the respective ssp strains R2S, T1S, X2B, or Z3A. Specifically, a ssp strain designated T1S comprises a genomic sequence which is at least 90%, or at least 95%, or at least 96%, or at least 97%, or at least 98%, or at least 99%, or 100% sequence identical to SEQ ID NO:19. Specifically, a ssp strain designated RS2 comprises a genomic sequence which is at least 90%, or at least 95%, or at least 96%, or at least 97%, or at least 98%, or at least 99%, or 100% sequence identical to SEQ ID NO:20.

Specifically, the sAOA are of one or more strains originating from human skin.

Specifically, the sAOA are isolated from an axenic culture, or from a co-culture with other microbes, such as with other AOA, in particular with other sAOA.

Specifically, the preparation is understood as being an sAOA preparation.

Specifically, the sAOA preparation may comprise the sAOA as live or inactivated microorganisms.

Specifically, the sAOA preparation may comprise a fraction of the sAOA, or comprise a fractionated preparation.

Specifically, the sAOA preparation comprises at least a probiotic fraction of the SAOA.

Specifically, the sAOA preparation comprises the sAOA in a probiotic formulation.

Specifically, any one or more or all of said ssp strains R2S, T1S, X2B, or Z3A are characterized by respective amoA sequences. Specifically, the amoA sequence is a wild-type sequence such as naturally occurring in a wild-type strain. Specifically, the amoA sequence is unique for the respective strain.

Specifically, the amoA gene is an archaeal gene encoding the alpha-subunit of the ammonia monooxygenase.

According to a specific aspect, said sAOA comprises an amoA gene comprising a sequence that is distinct from amoA gene sequences of any other Nitrosocosmicus strains, in particular when compared to Nitrosocosmicus strains that originate from soil or marine sources.

Specifically, each of said ssp strains R2S, T1S, X2B, or Z3A is characterized by a respective amoA sequence, which differs from any other AOA, in particular any other AOA of the genus Nitrosocosmicus,

Specifically, the difference is at least 13 nt over the full-length amoA sequence, or at least 13 nt within a region of the amoA sequence of at least 588 nt length (a partial sequence).

Specifically, the amoA gene sequence of ssp strain R2S comprises or consists of SEQ ID NO:1. Specifically, the respective amoA protein sequence of ssp strain R2S comprises or consists of SEQ ID NO:11.

Specifically, the amoA gene sequence of ssp strain T1S comprises or consists of SEQ ID NO:2. Specifically, the respective amoA protein sequence of ssp strain T1S comprises or consists of SEQ ID NO:12.

Specifically, the amoA gene sequence of ssp strain X2B comprises or consists of SEQ ID NO:3. Specifically, the respective amoA protein sequence of ssp strain X2B comprises or consists of SEQ ID NO:13.

Specifically, the amoA gene sequence of ssp strain Z3A comprises or consists of SEQ ID NO:4. Specifically, the respective amoA protein sequence of ssp strain Z3A comprises or consists of SEQ ID NO:14.

Specifically, each of the respective amoA sequences comprises a difference in the nucleotide sequence compared to the amoA sequence of any other sAOA of the genus Nitrosocosmicus.

A sequence alignment of partial amoA sequences and of respective amoA protein sequences with close relative strains were made with N._oleophilus_MY3 (SEQ ID NO: 5), N._arcticus_Kfb (SEQ ID NO:6), N._franklandianus_C13 (SEQ ID NO:7), and N._hydrocola_G61 (SEQ ID NO:8), resulting in a consensus nuceleotide sequence SEQ ID NO: 9; and in a consensus protein sequence SEQ ID NO:10.

The sequence comparison revealed which strains are the closest relative strains.

Specifically, the closest relative strain of ssp strain R2S is Ca. Nitrosocosmicus oleophilus strain MY3.

Specifically, the closest relative strain of ssp strain T1S is Ca. Nitrosocosmicus oleophilus strain MY3.

Specifically, the closest relative strain of ssp strain X2B is Ca. Nitrosocosmicus franklandianus.

Specifically, the closest relative strain of ssp strain Z3A is Ca. Nitrosocosmicus hydrocola.

Specifically, the amoA gene difference of the sAOA comprises at least 13 nt difference in the amoA gene compared to any one or more (or all) of Nitrosocosmicus oleophilus, Nitrosocosmicus arcticus, Nitrosocosmicus hydrocola or Nitrosocosmicus franklandianus strains, in particular when compared to a strain which is closely related to the sAOA. In specific case, the amoA gene of AOA that are closely related to any SAOA described herein, comprise at least 90%, or at least 95% sequence identity to the amoA gene of any such sAOA.

Specifically, the amoA gene difference is determined when comparing the full amoA gene sequence of Ca. Nitrosocosmicus arcticus or Ca. Nitrosocosmicus oleophilus strain MY3 or Ca. Nitrsosocosmicus hydrocola G61 or Ca. Nitrosocosmicus franklandianus C13, or when comparing a respective region comprising at least 588 consecutive nt within the full-length amoA gene sequence. Specifically, the regions compared to each other are spanning at least 588 consecutive nt and comprise highest sequence identity to each other.

Specifically, the difference in the amoA gene sequence is determined by pairwise comparison, in particular wherein a respective partial sequence spanning at least 588 nt is compared.

According to a specific aspect,
a) said sAOA is of Ca. Nitrosocosmicus ssp strain R2S, which comprises an amoA gene sequence comprising SEQ ID NO:1 that comprises at least 13 different nt compared to the amoA gene sequence SEQ ID NO: 5 originating from its closest relative Ca. Nitrosocosmicus oleophilus strain MY3, particularly 13 different nt in the compared region of 588 nt;
b) said sAOA is of Ca. Nitrosocosmicus ssp. Strain T1S, which comprises an amoA gene sequence comprising SEQ ID NO:2 that comprises at least 25 different nt compared to the amoA gene sequence SEQ ID NO:5 originating from its closest relative Ca. Nitrosocosmicus oleophilus, particularly 25 different nt in the compared region of 588 nt;
c) said sAOA is of Ca. Nitrosocosmicus ssp strain X2B, which comprises an amoA gene sequence comprising SEQ ID NO:3 that comprises at least 34 different nt compared to the amoA gene sequence SEQ ID NO:7 originating from its closest relative Ca. Nitrosocosmicus franklandianus;
d) said sAOA is of Ca. Nitrosocosmicus ssp strain Z3A, which comprises an amoA gene sequence comprising SEQ ID NO:4 that comprises at least 30 different nt compared to the amoA gene sequence SEQ ID NO:8 originating from its closest relative Ca. Nitrosocosmicus hydrocola.

Ca. Nitrosocosmicus strain R2S and Ca. Nitrosocosmicus strain T1S, as well as strains X2B and Z3A, are sAOA that belong to the family of Nitrososphaeraceae of the phylum Thermoproteota of the domain Archaea. Their morphological appearance is similar to described strains of the genus Nitrosocosmicus (e.g., Nitrosocosmicus franklandianus and N. oleophilus) and slightly different from that of Nitrososphaera viennensis and related strains of the genus Nitrososphaera.

According to a specific aspect, the sAOA preparation comprises further ammonia oxidizing microorganisms, such as AOA and/or AOB.

According to a specific aspect, the sAOA preparation comprises further AOA originating from environmental sources, preferably from human skin, soil or marine sources, preferably AOA of the family of Nitrosospheraceae, preferably selected from one or more of:
a) the genus Nitrososphaera, preferably Nitrososphaera viennensis, preferably Nitrososphaera viennensis EN76, Nitrososphaera viennensis EN123, or Nitrososphaera viennensis KFJ;
b) the genus Nitrosocosmicus, preferably Nitrosocosmicus oleophilus, Nitrosocosmicus arcticus, Nitrosocosmicus hydrocola or Nitrosocosmicus franklandianus, preferably Ca. Nitrosocosmicus oleophilus strain MY3, Ca. Nitrosocosmicus sp. (Mir11), Nitrosocosmicus franklandianus (formerly franklandus) C13, or Nitrosocosmicus hydrocola G61.

According to a specific aspect, the sAOA are obtained by a method of growing the sAOA in a cell culture and isolating the sAOA from the cell culture, optionally wherein the sAOA are inactivated and/or fractionated.

Specifically, the cell culture is a culture of the sAOA in controlled bioreactor systems, such as in liquid culture or fermenters.

Specifically, the culture is an sAOA monoculture or co-culture with other AOA, such as e.g., with any kind of AOA, or with other sAOA (e.g., a co-culture with more than one different sAOA strains or species).

Specifically, the sAOA preparation is a preparation of viable (i.e., live) or inactivated sAOA.

Specifically, the sAOA preparation is a preparation of fractionated sAOA, comprising at least any one or more of archaeal proteins (such as e.g., surface proteins), cellular components, extracellular or intracellular metabolites.

Specifically, the sAOA preparation is provided as a storage-stable preparation, preferably storage-stable at room temperature, for at least 1, 2, 3, 4, 5, 6, 7, 8, 9, 10, 11, or 12 months.

The invention further provides for a composition comprising the sAOA preparation described herein, and a cosmetic carrier.

Specifically, the cosmetic composition is provided in the form of a cream, emulsion, gel, ointment, lotion, spray, aerosol, foam, solution, dry powder, bath salt, bath ball, tablet, shampoo, conditioner, deodorant, soap, skin gel, lip balm, facial mask, or makeup.

Specifically, the cosmetic composition is packaged for single or multiple use.

The invention further provides for the use of the sAOA preparation described herein for topical application to a human body. Topical application typically refers to the surface of the skin, a wound, and/or mucosal cells or tissues (e.g., alveolar, buccal, dermal, lingual, masticatory, or nasal mucosa, etc.). Topical application comprises e.g., the application to body surfaces, such as to skin, hair or mucosa.

Specifically, the sAOA preparation is used for any one or more of the following: regulating the microbiome, moisturizing, purifying, achieving anti-stinging, soothing, anti-aging, or improving barrier function of skin, hair or mucosa.

The invention further provides for the medical use of the sAOA preparation described herein.

Specifically, the sAOA is provided herein for use as a medicament.

Specifically, the sAOA is used in a method of treating a subject in need of medical treatment. Specifically, the treatment comprises administering an effective amount of the sAOA preparation, such as an amount that is suitable used for medical management of a subject with the intent to cure, ameliorate, stabilize, reduce the incidence or prevent a disease, pathological condition, or disorder, which individually or together are understood as “disease condition”.

Specifically, the effective amount of sAOA is between 1×10³ cells/mL and about 1×10¹⁴ cells/mL, such as an amount used per dose or formulation ready for application or use e.g., in a cream for skin treatment, or for mucosal use.

Specifically, treatment is provided for a variety of disease conditions, such as e.g., skin-related disease or disorder. Specifically, a skin-related disease or disorder refers to an abnormal skin condition caused by inflammatory, allergic or autoimmune reactions. Typical symptoms of skin related diseases or disorders referred to herein include, for example, raised bumps that are red or white, a rash (which might be painful or itchy), scaly or rough skin, peeling skin, open sores or lesions, and dry, cracked skin.

Specifically, a subject suffering from a dermatologic disease or disorder is treated, such as e.g., acne, psoriasis, atopic dermatitis, skin inflammation or skin redness.

Inflammatory diseases of the skin include, for example, dermatitis, bacterial and viral skin inflammation, and acne. Specifically, dermatitis comprises e.g., atopic dermatitis, contact dermatitis, such as irritant contact dermatitis and allergic contact dermatitis, allergic dermatitis, pruritic dermatitis, solar (UVB-induced) dermatitis and chemical-induced dermatitis. Specifically, atopic dermatitis, also called eczema, is typically caused by a defect of stratum corneum, which is a protective wall located in the outermost part of the skin, caused by hereditary, environmental, or immunological factors and is exacerbated in arid climate. The symptoms of atopic dermatitis include e.g., severe pruritus (itch), xeroderma, eruption or oozing of the skin, boils and scale like skin (scaly skin). Specifically, the skin-related disease or disorder includes secondary skin infections caused by dermatitis, such as e.g., caused by scratching.

The invention further provides for a pharmaceutical preparation comprising the sAOA preparation described herein, and a pharmaceutically acceptable carrier, preferably formulated in the form of a cream, emulsion, gel, ointment, lotion, spray, aerosol, pellet or dry powder.

Specifically, the pharmaceutical preparation is provided for use in the medical treatment of a human subject as further described herein.

Specifically, the pharmaceutical preparation is administered to body surfaces, preferably skin, hair or mucosa, in an effective amount to modulate the subject's microbiome.

The invention further provides for a method of producing a, sAOA preparation described herein, comprising:
a) culturing the sAOA in a cell culture,
b) separating the sAOA from the cell culture, and
c) formulating the sAOA preparation;
optionally wherein the sAOA are inactivated and/or fractionated.

Specifically, a fraction of sAOA is separated from the cell culture, preferably by centrifugation and/or filtration.

Specifically, sAOA centrifugation is performed in a table top centrifuge (e.g., Eppendorf 5415R Refrigerated Centrifuge; for example, at 16.000× g, for 30 min at 4° C. The pellet is resuspended in a small volume of FWM (fresh water medium) as needed.

Specifically, sAOA filtration is done on nitrocellulose filters with 0.2 μm pore size (e.g., MCE membrane MF Millipore). The recovered cells are washed off with low volumes of FWM.

Specifically, sAOA inactivation is by heat inactivation and/or inactivation using organic solvents such as ethanol.

Specifically, heat inactivation is performed by treating cells at at least 70° C. for at least 2 hours.

Specifically, inactivation using organic solvents is performed by adding ethanol or isopropanol to the cell suspension reaching a final concentration of 10% (v/w).

Specifically, sAOA fractionation is by separating a fraction comprising archaeal components, such as an extract, cellular components, or portion, from another fraction. Separation may be carried out by biological, physical and/or chemical means. Specific fractions may be obtained from tyndallized, or sonicated or otherwise inactivated archaea, using well-known techniques, or lysates of the archaeal strain, or extracts of the archaeal strain. Specific fractions may include proteins, polysaccharides, cellular materials, or metabolites such as metabolic bioproducts generated by the archaeal strain.

Therefore, the present invention also refers to sAOA preparations obtainable or obtained by any such production methods described herein including e.g., preparations comprising inactivated and/or fractionated sAOA.

### FIGURES

FIG. 1. Venn diagram of orthologous proteins between closed Nitrosocosmicus genomes. Proteins were predicted using Prodigal v2.6.31 followed by orthologous prediction with OrthoFinder v2.5.42. In brackets: total number of orthologs in each genome.

FIG. 2. Phylogenetic tree of partial amoA genes. amoA genes were aligned using MAFFT v7.515 ³(L-INS-i algorithm) and trimmed based on the shortest sequence keeping a total of 588 nucleotide sites. The tree was reconstructed using IQTREE v2.2.24 under the model HKY+F+I of sequence evolution.

FIG. 3. Pairwise comparison of number of point mutations between partial amoA genes in Nitrosocosmicus (over 588 nt). Total number of differences were estimated using Geneious Prime® 2022.2.2. Higher color intensity indicates lower number of differences between sequences.

FIG. 4. Alignment of partial amoA genes. amoA genes were aligned using MAFFT v7.515 (L-INS-i algorithm) 3 and trimmed based on the shortest sequence keeping a total of 588 nucleotide sites. Nucleotide differences are highlighted in black background. B) Dot representation of the alignment. Differences to the consensus sequence are highlighted. Wild-type amoA genes SEQ ID NO:1-8; consensus sequence: SEQ ID NO:9.

FIG. 5. Alignment of AmoA proteins. AmoA proteins were aligned using MAFFT v7.515 (L-INS-i algorithm) and trimmed based on the shortest sequence keeping a total of 196 amino acid sites. Amino acid differences are highlighted in black background. B) Dot representation of the alignment. Differences to the consensus sequence are highlighted. Wild-type AmoA proteins SEQ ID NO:11-18; consensus sequence: SEQ ID NO: 10.

FIG. 6. Phylogenomic reconstruction based on concatenation of 33 protein families present in the genomes of the dataset.

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (4th Ed.), Vols. 1-3, Cold Spring Harbor Laboratory Press (2012); Krebs et al., “Lewin's Genes XI”, Jones & Bartlett Learning, (2017), and Murphy & Weaver, “Janeway's Immunobiology” (9th Ed., or more recent editions), Taylor & Francis Inc, 2017.

The subject matter of the claims specifically refers to artificial products or methods employing or producing such artificial products, which may be variants of native (wild-type) products. Though there can be a certain degree of sequence identity to the native structure, it is well understood that the materials, methods and uses described herein, e.g., specifically referring to isolated nucleic acid sequences, amino acid sequences, fusion constructs, expression constructs, transformed host cells and modified proteins, are “man-made” or synthetic, and are therefore not considered as a result of “laws of nature”.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

Specific terms as used throughout the specification have the following meaning.

The sAOA preparations described herein are specifically used as probiotics.

The term “probiotics” or “probiotic” as used herein, is understood in the following way. Live microorganisms or inactivated preparations of such microorganisms, when administered or applied to a subject in adequate amount, are understood as having a probiotic effect if conferring benefits to the health of the subject. The term “probiotics” shall also comprise “prebiotic” substances. The sAOA preparations, or fractions thereof described herein may comprise prebiotic substances, in particular prebiotics that have “archaeogenic” properties, which are herein understood as improving the microbiome, in particular the microbiome of sAOA of human beings.

Likewise, the sAOA preparations described herein can be used as probiotics when formulated as a pharmaceutical preparation, such as a drug or drug product.

The use of probiotics or prebiotics has been proposed in cosmetics. The skin is a complex barrier organ that has a symbiotic relationship between microbial communities and host tissue via complex signals provided by the innate and the adaptive immune systems. It is constantly exposed to various endogenous and exogenous factors-physical, chemical, bacterial and fungal, as well as the effects of the hormonal disorders, which affect this balanced system potentially leading to inflammatory skin conditions comprising infections, allergies or autoimmune diseases. Therefore, the screening of effective means of correcting and/or maintaining the human normoflora for the preservation of healthy skin microbiome is considered important.

Typical prebiotic substances as used in the sAOA preparations and formulations described herein, may include e.g., compounds that induce the growth or activity of beneficial microorganisms such as archaea. Specifically preferred are prebiotic substances comprised in AOA or fractions thereof.

It is well known that probiotics and prebiotics are helpful for specific disorders in the human body. Probiotic therapy can have particular benefits in accelerating wound healing, in preventing and treating the skin diseases including eczema, atopic dermatitis, acne, allergic inflammation or skin hypersensitivity, UV-induced skin damage and cosmetics products. Therefore, the sAOA described herein may comprise the sAOA or fractions thereof (e.g., lysates) as components of cosmetic products with the promise of “rebalancing” the microbial community that live in or on the human body and delivering healthier, more radiant-looking skin. The parameters as the type of sAOA probiotic (or prebiotic) composition, the form in which it is added to the formulation (living or inactivated sAOA, lysates, etc.) and the recommended concentrations of these ingredients in cosmetic products that are safe and effective can be determined as well-known in the art, considering that there is widespread use of probiotic cosmetic products.

The sAOA preparation formulated for cosmetic use may be provided as cosmetic preparations which comprise components as selected for cosmetic formulation such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid.

The term “effective amount” with respect to a probiotic effect as used herein, shall refer to an amount (in particular a predetermined amount) that has a proven probiotic effect. The amount is typically a quantity or activity sufficient to, when applied to a surface or administered to a subject effect beneficial of desired results, and, as such, an effective amount or synonym thereof depends upon the context in which it is being applied.

The concentration of active substances in the sAOA preparation described herein is preferably between 0.1 and 10% biomass (w/w), in particular wherein biomass is biomass originating from sAOA.

An effective amount of a pharmaceutical preparation or drug is intended to mean that amount of a compound that is sufficient to treat, prevent or inhibit a disease, disease condition or disorder. Such an effective dose specifically refers to that amount of the compound sufficient to result in healing, prevention or amelioration of conditions related to diseases or disorders described herein.

In the context of disease, effective amounts (in particular prophylactically or therapeutically effective amounts) of the sAOA preparation described herein, are specifically used to treat, modulate, attenuate, reverse, or affect a disease or condition that benefits from its probiotic effect. The amount of the compound that will correspond to such an effective amount will vary depending on various factors, such as the given drug or compound, the formulation, the route of administration, the type of disease or disorder, the identity of the subject or host being treated, the assessment of the medical situations and other relevant factors, but can nevertheless be routinely determined by one skilled in the art.

A treatment or prevention regime of a subject with an effective amount of the sAOA preparation described herein may consist of a single application or administration, or alternatively comprise a series of applications and administrations, respectively. For example, the sAOA preparation may be used at least once a month, or at least once a week, or at least once a day. However, in certain cases of an acute phase e.g., upon suspected or confirmed disorder, or after a disorder has been determined, the sAOA preparation may be used more frequently e.g., 1-10 times a day.

Specifically, a combination therapy is provided which includes treatment with the sAOA preparation described herein and standard therapy of the respective disease or disorder. Doses may be applied in combination with other active agents such as anti-inflammatory drugs, or antibiotics.

Treatment can be combined with active agents of standard treatment, preferably wherein a pharmaceutical preparation is administered before, during (e.g., by co-administration or in parallel), or after administration of said active agents.

Specifically, the sAOA preparation described herein can be combined with one or more other active substances.

Specifically, the sAOA preparation is combined with an anti-inflammatory agent such as steroidal anti-inflammatory drugs, glucocorticoids and nonsteroidal anti-inflammatory drugs (NSAID's). Suitable NSAID's include, but are not limited to ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid and celecoxib. Suitable steroidal anti-inflammatory agents include, but are not limited to, corticosteroids such as synthetic glucocorticoids. Specific examples are fluticasone, COX-2 inhibitors, ibuprofen, hydroxychloroquine, heparin, LMW heparin, hirudine, or immunosuppressants, such as azathioprine, cyclosporin A, or cyclophosphamide.

Specifically, the sAOA preparation is combined with an antibiotic such as a beta lactam antibiotic, an aminoglycoside antibiotic, an ansamycin, a carbacephem, a carbapenem, a cephalosporin, a glycopeptide, a lincosamide, a lipopeptide, a macrolide, a monobactam, a nitrofuran, an oxazolidinone, a polypeptide, a sulfonamide, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Mupirocin, Platensimycin, Quinupristin/Dalfopristin, Thiamphenicol, Tigecycline, Tinidazole, Trimethoprim, Teixobactin, Malacidins, Halicin, clindamycin, vancomycin, metronidazole, fusidic acid, thiopeptides, fidaxomicin, quinolons, tetracyclins, omadacycline, rifamycin, kibdelomycin, oxazolidinone, ketolides, thiazolides, amixicile, teicoplanin, ramoplanin, oritavancin, lantibiotics, capuramycin, surotomycin, thuricin, endolysin, avidocin CD, cadazolid, ramizol, defensins, ridinilazole, medium-chain fatty acids, phages, berberine, lactoferrin.

According to a specific aspect, the sAOA preparation is combined with an antimicrobial substance that is not effective against Archaea, but effective on pathogenic bacteria or yeast.

The length of the treatment period depends on a variety of factors, such as the severity of the disease, either acute or chronic disease, the age of the patient, and the concentration of the sAOA preparation. It will also be appreciated that the effective dosage used for the treatment or prophylaxis may increase or decrease over the course of a particular treatment or prophylaxis regime. Changes in dosage may result and become apparent by standard diagnostic assays known in the art.

According to a specific aspect, a medicinal product or pharmaceutical composition described herein contains an effective amount of the sAOA preparation as defined herein. The preparation described herein may be provided for single or multiple dosage use.

Unit-dose or multi-dose containers may be used, for example, sealed ampoules and vials, or multi-use sprays, and may be stored comprising a liquid, semi-liquid, or dry phase, e.g., in a freeze-dried (lyophilized) condition requiring only the addition of the sterile liquid carrier, for example water for injection, immediately prior to use. Preferred unit dosage formulations are those containing a daily dose or unit daily sub-dose, or multiple doses, of the sAOA preparation.

The term “single-dose” as used herein is understood in the following way. A single-dose or amount for single-use is the amount intended for administration that is meant for use in a single subject, such as a patient, either human or animal for a single case/procedure/administration. Packages comprising the single-dose are typically labelled as such by the manufacturer. The single-dose amount is specifically understood as a daily dose for an individual, like a child or adult, to provide an effective amount.

The medicinal product or pharmaceutical composition described herein is specifically provided as human medicinal product or pharmaceutical composition.

Medicinal products are understood as substances that are used to treat diseases, to relieve complaints, or to prevent such diseases or complaints in the first place. The substances can act both within or on the body.

The sAOA preparation formulated for pharmaceutic use may be provided as pharmaceutical preparations as further described herein. Specific formulations may comprise components such as, for example, selected from proteins, carbohydrates, fats, further probiotics, prebiotics, enzymes, vitamins, immune modulators, minerals, amino acids, coccidiostats, acid-based products, drugs, medicines, and combinations thereof.

The medicinal product or pharmaceutical composition described herein preferably contains one or more pharmaceutically acceptable auxiliaries and is in a pharmaceutical form which allows the active pharmaceutical compound to be administered with high bioavailability. Suitable auxiliaries may be, for example, based on cyclodextrins. Suitable formulations might for example incorporate synthetic polymeric nanoparticles formed of a polymer selected from the group consisting of acrylates, methacrylates, cyanoacrylates, acrylamides, polylactates, polyglycolates, polyanhydrates, polyorthoesters, gelatin, albumin, polystyrenes, polyvinyls, polyacrolein, polyglutaraldehyde and derivatives, copolymers and mixtures thereof.

Specific medicinal products or pharmaceutical compositions described herein comprise the sAOA preparation and a pharmaceutically acceptable carrier.

A “pharmaceutically acceptable carrier” refers to an ingredient in a formulation for medicinal or medical use, other than an active ingredient, which is nontoxic to a subject. A pharmaceutically acceptable carrier includes, but is not limited to, a buffer, excipient, stabilizer, or preservative, and in particular saline, phosphate buffered saline, dextrose, glycerol, ethanol, and the like.

The sAOA preparation as used herein can be formulated with conventional carriers and excipients, which will be selected in accordance with ordinary practice.

Pharmaceutically acceptable carriers generally include any and all suitable solvents, dispersion media, coatings, antiviral, antibacterial and antifungal agents, isotonic and absorption delaying agents, and the like that are physiologically compatible with an antiviral small molecule compound or related composition or combination preparation described herein.

According to a specific aspect, the sAOA preparation can be combined with one or more carriers appropriate a desired route of administration. The sAOA preparation may be e.g., admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, the sAOA preparation may be dispersed or dissolved in saline, water, polyethylene glycol, propylene glycol, carboxymethyl cellulose colloidal solutions, ethanol, corn oil, peanut oil, cotton seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well-known in the art.

Compounds as described herein may be provided in controlled release pharmaceutical (“controlled release formulations”) in which the release of the sAOA preparation is controlled and regulated to allow less frequency dosing or to improve the pharmacokinetic or toxicity profile of a given active ingredient.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Additional pharmaceutically acceptable carriers are known in the art and described in, e.g., Remington: The Science and Practice of Pharmacy, 22^{nd} revised edition (Allen Jr, L V, ed., Pharmaceutical Press, 2012). Liquid formulations can be solutions, emulsions or suspensions and can include excipients such as suspending agents, solubilizers, surfactants, preservatives, and chelating agents.

The preferred preparation is in a ready-to-use, storage stable form, with a shelf-life of at least one or two years.

The term “formulation” as used herein refers to a preparation ready-to-use in a specific way. Specifically, compositions described herein comprises the sAOA preparation, and a pharmaceutically acceptable diluent, carrier or excipient.

According to a specific aspect, formulations are provided comprising pharmaceutically acceptable vehicles for topical, mucosal (e.g., nasal, peroral), or parenteral administration. Administration may particular be dermal. Also, the present disclosure includes such preparations, which have been lyophilized and which may be reconstituted to form pharmaceutically acceptable formulations for administration.

Specific medicinal products or pharmaceutical compositions described herein are formulated for topical route e.g., onto biological surfaces, including e.g., mucosa or skin. Pharmaceutical carriers suitable for facilitating such means of administration are well known in the art.

To administer the sAOA preparation by any route other than parenteral administration, it may be necessary to coat the active agent with, or co-administer the active agent with, a material to prevent its inactivation through enzymatic degradation and/or digestion. For example, an appropriate carrier may be used, for example, liposomes, or a diluent. Pharmaceutically acceptable diluents include saline and aqueous buffer solutions.

The sAOA preparation can be orally administered, for example, with an inert diluent or an assimilable or edible carrier. For example, a preparation may be enclosed in a hard- or soft-shell gelatin capsule, or compressed into tablets. For oral therapeutic administration, the sAOA preparation may be incorporated with excipients and used in the form of ingestible tablets, buccal tablets, troches, capsules, elixirs, suspensions, syrups, wafers, and the like. The percentage of the compound in the compositions and preparations may, of course, be varied. The amount of the sAOA preparation in such therapeutically useful compositions is such that a suitable dosage will be obtained.

Tablets will contain excipients, glidants, fillers, binders, disintegrants, lubricants, flavors and the like. Granules may be produced using isomaltose. It is furthermore preferred to provide for a preparation formulated to act at the site of the mucosa, e.g. at mucosal sites (such as nose, mouth, eyes, esophagus, throat, lung), e.g. locally without systemic action. Aqueous formulations are prepared in sterile form, and when intended for delivery by other than oral administration generally will be isotonic.

The term “mucosal” with respect to administration or application or else mucosal use of a preparation for treating a subject or a respective formulation, refers to administration via the mucosal route, including systemic or local administration, wherein an active ingredient is taken up by contact with mucosal surfaces. This includes nasal, pulmonary, oral, or peroral administration and formulations, e.g., liquid, syrup, lozenge, an eye drop, tablet, spray, powder, instant powder, granules, capsules, cream, gel, drops, suspension, or emulsion.

Peroral formulations may include liquid solutions, emulsions, suspensions, and the like. The pharmaceutically acceptable vehicles suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the sAOA preparation include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose, or glidants, lubricants, sweeteners, colorants, antioxidants and flavoring agents.

The sAOA preparation can also be administered topically to a subject, e.g., by the direct laying on or spreading of a composition containing same on the epidermal or epithelial tissue of the subject, or transdermally via a “patch”. Such compositions include, for example, lotions, creams, solutions, gels and solids. These topical compositions may comprise an effective amount, usually from about 0.1 to 10% biomass (w/w), in particular wherein biomass is biomass originating from sAOA. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

Pharmaceutical compositions suitable for injectable use include sterile aqueous solutions (in particular where the compounds or pharmaceutically acceptable salts are water soluble) or dispersions and sterile powders for the extemporaneous preparation of sterile injectable solutions or dispersion. In particular, the composition is specifically sterile and fluid to the extent that easy syringability exists; it is stable under the conditions of manufacture and storage and preserved against the contaminating action of microorganisms such as bacteria and fungi.

Suitable pharmaceutically acceptable vehicles include, without limitation, any non-immunogenic pharmaceutical adjuvants suitable for oral, parenteral, nasal, mucosal, transdermal, intravascular (IV), intraarterial (IA), intramuscular (IM), and subcutaneous (SC) administration routes, such as phosphate buffer saline (PBS).

The term “sequence identity” of a variant as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or genes by detecting excess similarity, and statistically significant similarity that reflects common ancestry.

“Percent (%) amino acid sequence identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

For purposes described herein, the sequence identity between two amino acid sequences is determined using the NCBI BLAST program version BLASTP 2.8.1 with the following exemplary parameters: Program: blastp, Word size: 6, Expect value: 10, Hitlist size: 100, Gapcosts: 11.1, Matrix: BLOSUM62, Filter string: F, Compositional adjustment: Conditional compositional score matrix adjustment.

For pairwise protein sequence alignment of two amino acid sequences along their entire length the EMBOSS Needle webserver (https://www.ebi.ac.uk/Tools/psa/emboss_needle/) was used with default settings (Matrix: EBLOSUM62; Gap open: 10; Gap extend: 0.5; End Gap Penalty: false; End Gap Open: 10; End Gap Extend: 0.5). EMBOSS Needle uses the Needleman-Wunsch alignment algorithm to find the optimum alignment (including gaps) of the two input sequences and writes their optimal global sequence alignment to file.

“Percent (%) identity” with respect to a nucleotide sequence is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

For purposes described herein (unless indicated otherwise), the sequence identity between two amino acid sequences is determined using the NCBI BLAST program version BLASTN 2.8.1 with the following exemplary parameters: Program: blastn, Word size: 11, Expect threshold: 10, Hitlist size: 100, Gap Costs: 5.2, Match/Mismatch Scores: 2, −3, Filter string: Low complexity regions, Mark for lookup table only.

The term “subject” as used herein shall refer to a warm-blooded mammalian, particularly a human being or a non-human animal, including e.g., dogs, cats, rabbits, horses, cattle, and pigs. Specifically, the sAOA preparation described herein is provided for human medical or veterinary use. The subject may be a patient suffering from a disease or disease condition.

The term “patient” includes human and other mammalian subjects that receive either prophylactic or therapeutic treatment. The term “treatment” is thus meant to include both prophylactic and therapeutic treatment. The term “patient” as used herein always includes healthy subjects, if receiving prophylactic treatment.

Specifically, the term “prophylaxis” refers to preventive measures which is intended to encompass prevention of the onset of pathogenesis or prophylactic measures to reduce the risk of pathogenesis.

The term “therapy” as used herein with respect to treating subjects refers to medical management of a subject with the intent to cure, ameliorate, stabilize, reduce the incidence or prevent a disease, pathological condition, or disorder, which individually or together are understood as “disease condition”. The term includes active treatment, directed specifically toward the improvement of a disease condition, prophylaxis directed specifically toward the prevention of a disease condition, and also includes causal treatment directed toward removal of the cause of the associated disease condition. In addition, this term includes palliative treatment designed for the relief of symptoms rather than the curing of the disease condition, and further curing a disease condition directed to minimizing or partially or completely inhibiting the development of the associated disease condition, and supportive treatment employed to supplement another specific therapy directed toward the improvement of the associated disease condition.

Therefore, the present disclosure is based on sAOA preparations obtained from new sAOA strains that were isolated and characterized. The strains were obtained by enrichment in cultures of healthy human skin samples. The strains were isolated and further characterized by genomic analysis. The genomes of both strains have been assembled after sequencing from an enrichment culture.

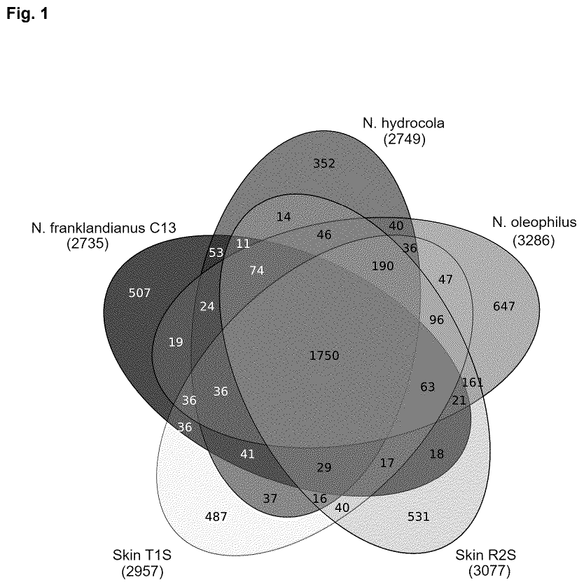

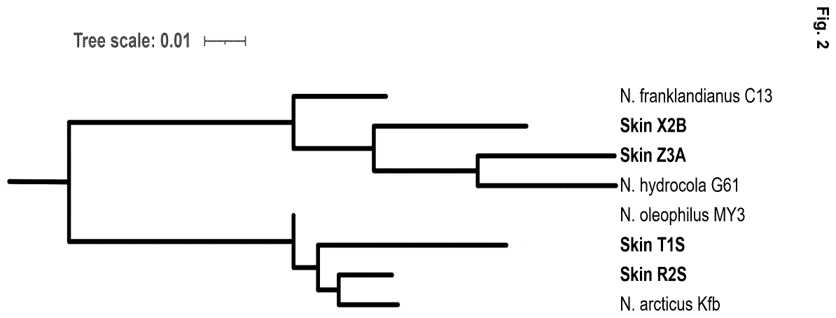

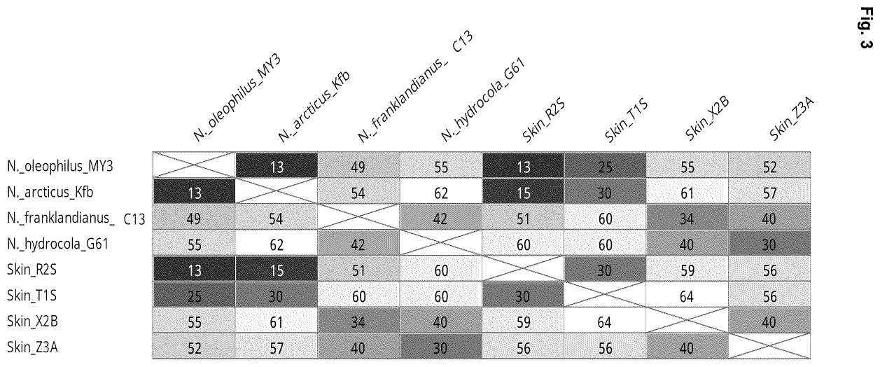

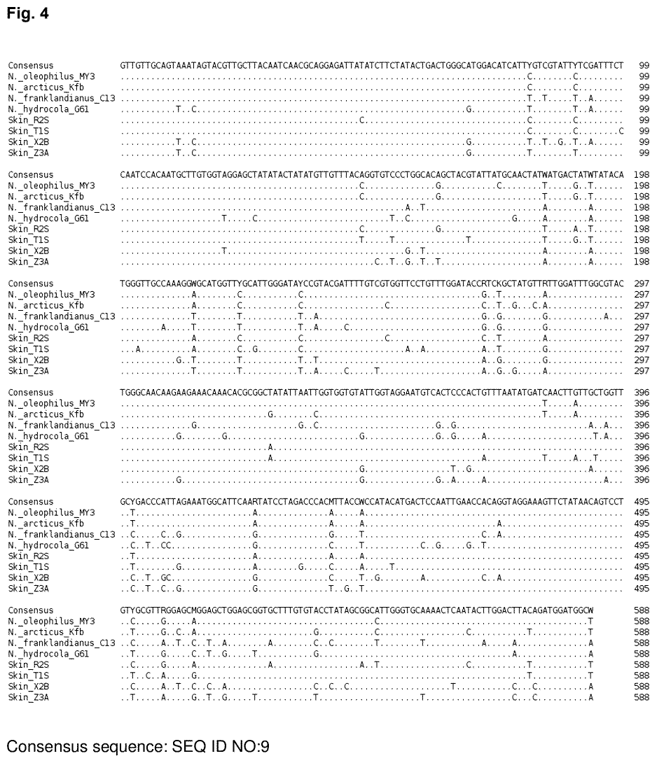

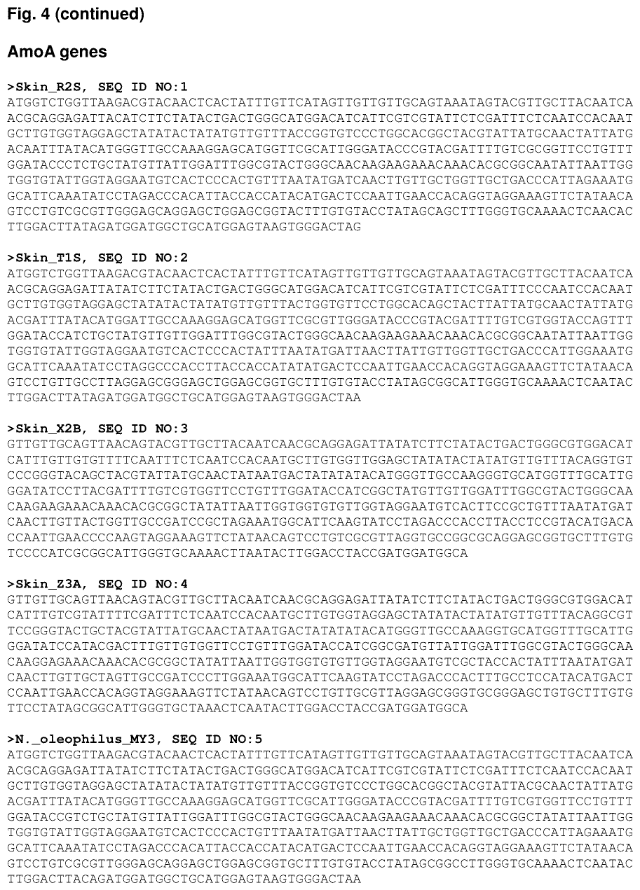

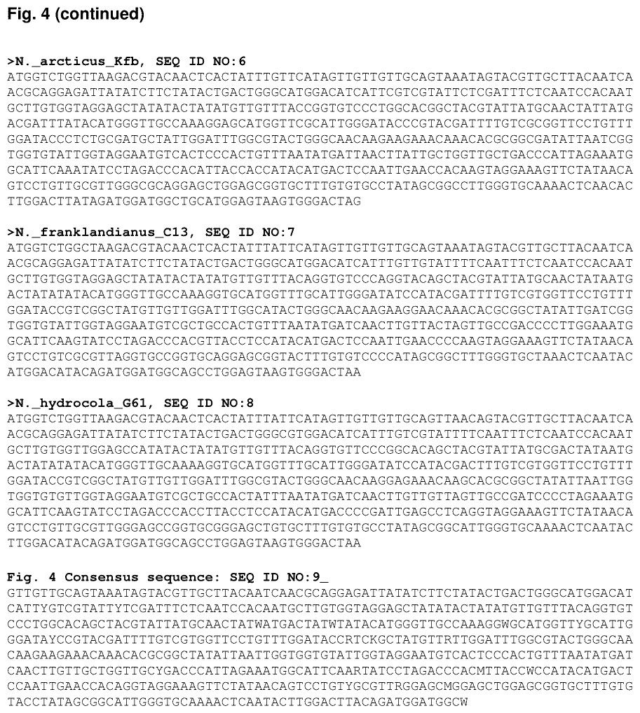

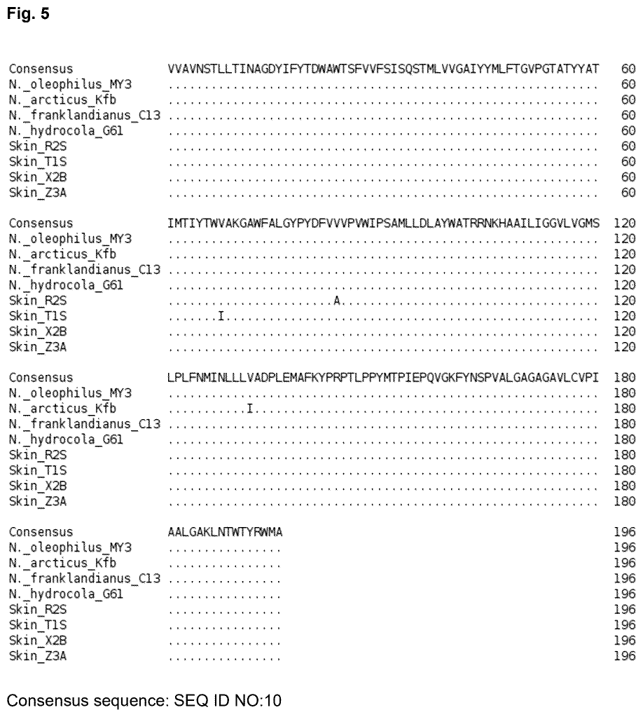

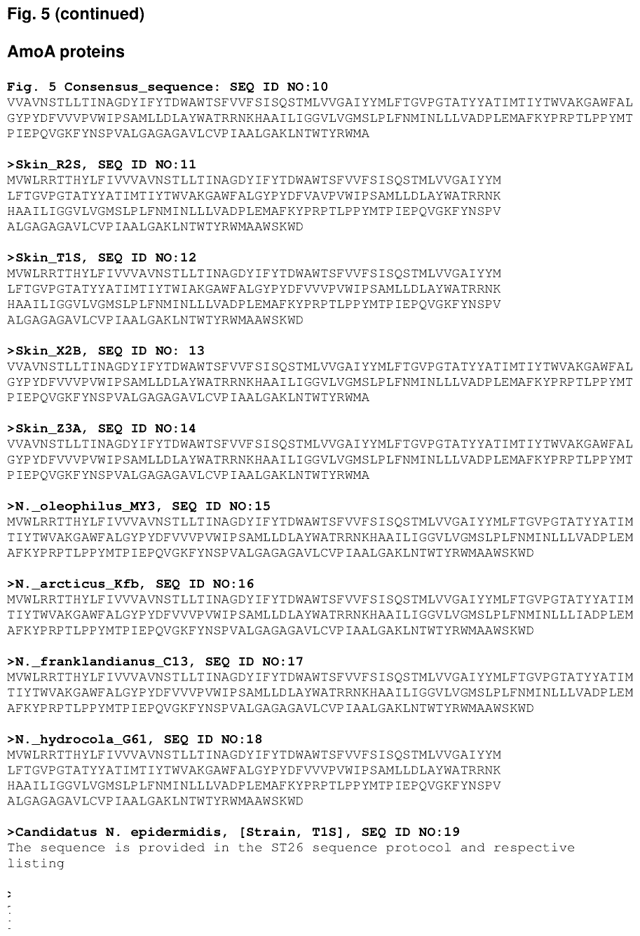

FIG. 1 displays a Venn diagram that shows the overlap of orthologous groups (=genes) among the two skin-derived genome bins and the three most closely related strains of the genus Nitrosocosmicus. While 1580 genes are shared among all five strains, Skin bin_1 contains 573 and Skin bin_2 contains 579 unique genes. The two respective organisms from human skin are therefore justified to be placed into a separate species, which are herein tentatively named: Candidatus Nitrosocosmicus epidermidis.

The identification of new isolates which are sAOA isolated from human healthy skin, provides for new products and treatments, in particular skin treatments.

Evidence was provided that archaea were thriving on healthy human skin into laboratory culture, and could be propagated. Alike AOA, the sAOA gain energy by oxidizing ammonia or urea (both produced in sweat) to nitrite (commonly referred to as ammonia oxidizing archaea, or AOA) [20, 21]. As a side product, they also release NO (nitric oxide) [22]. Both nitrite and NO are important signaling molecules with a broad range of physiological functions such as inflammation (activating macrophages), vasodilation, wound healing and tumor cell response [23-26]. These organisms are thus prone to have beneficial properties under (immuno-) pathological conditions. Comparable preparations are derived from ammonia oxidizing bacteria (AOB) isolated from soil (not skin) and sold by AOBiome (Cambridge, MA, USA).

The findings suggest that sAOA which are native AOA sourced from human skin, can be produced and can be applied on human irritated, compromised and diseased skin and enable/accelerate reverting into a healthy state in a sustainable way and with improved retention chances. Moreover, not a single archaeal representative has ever been identified to be pathogenic or to carry pathogenic traits, making archaea perfect candidates for probiotic-based medical applications (“Archaeobiotics”; [14, 27]). Applying archaea as skin probiotics thus represents a profound strategy for microbiome therapy. Pioneer small-scale studies on modulation of the skin microbiome by applying endogenous and exogenous bacterial strains associated with healthy states have generally shown positive initial results [12, 28-30]. The sAOA preparation described herein is likewise expected to have applications in skin care as it would in general promote skin homeostasis and could also reduce e.g. skin aging, or pigmentation by maintaining skin pH, detoxifying harmful compounds of secondary metabolism of other microbes and putatively improving moisture retention [28].

SAOA preparations described herein have a high potential in applications as probiotics on skin for three major reasons:
(i) The general robustness of archaea (as opposed to bacteria) and their capacity to survive unfavorable environmental conditions and fluctuations, such as high/low salt (in sweat/after washing), low or fluctuating ammonia and urea availability, all will improve their survival on skin and thus result in a more sustainable product [32, 33];
(ii) the low nutrient demands of AOA (oligotrophic lifestyle) will equally support their maintenance on skin [34, 35];
(iii) no single pathogenic representative is known from archaea. This is an important aspect for safety considerations. Nevertheless, the interactions of gastrointestinal archaea with the human immune system are starting to be addressed (reviewed in [36].

The beneficial role of the applied sAOA would be mediated by:
(i) targeted application and production of NOx (nitric oxide and nitrite), the effective delivery of which is still a challenge in pharmacokinetics [37];
(ii) the modulation of the immune response, in a manner similar to other microbes including AOB [38];
(iii) the putative production of novel antibiotics as well as detoxification mechanisms, since their genomes reveal enriched capacities in these processes [32, 39];
(iv) their autotrophic lifestyle which makes them a keystone community member, as they produce and supply vitamins, co-factors and other essential nutrients to other commensals, thereby stabilizing a healthy microbial community as has been shown in other ecosystems [40].

Ammonia oxidizing bacteria (AOB) and respective strains are described in WO2019104136A1. Ongoing clinical studies show promising preliminary results using the AOB. It is described that such effect was through their production of nitrite and nitric oxide (NOx). Thus, ammonia oxidizing microorganisms are described with a potential to assist in wound closure, improve atopic skin conditions and modulate immune responses [29, 38, 42, 43]

The important advantage of sAOA over AOB is the fact that archaea are naturally found on humans as shown by molecular biological studies [15, 17, 44], just like some probiotics used to improve gut health stem from healthy humans (e.g. certain strains of Bifidobacterium or lactobacilli). In contrast, the AOB strain as described in the prior art has, to the present knowledge, not been detected on human surfaces.

Specific sAOA isolates described herein stem from a healthy child. As such, the respective sAOA preparation will not only be able to exert the same positive effects as the bacteria used by AOBiome, but a superior performance is expected for the following reasons:
(i) it is adapted to human skin, thus longer retention times and more successful engraftment are expected;
(ii) AOA dominate over AOB in their activity, particularly in natural more ‘extreme’ environments, such as acidic soils, colder soils (arctic), and warmer soils (closer to the temperature of humans) [45-48] (iii) AOA have a more efficient CO₂ fixation pathway than AOB, enabling them to maintain stable populations on low carbon availabilities [49, 50];
(iv) genomes of related AOA are enriched in detoxification mechanisms, increasing their survival potential [32]; the same might be true for skin AOA.
(v) as a future perspective: isolation of different strains specific to human subpopulations [51] will be possible and will allow to tailor the product to specific markets and patients (personalized medicine).

The foregoing description will be more fully understood with reference to the following examples. Such examples are, however, merely representative of methods of practicing one or more embodiments of the present invention and should not be read as limiting the scope of invention.

### EXAMPLES

### Example 1: Characterisation of Ca. Nitrosocosmicus unguis Strain R2S and Ca. N. epidermidis Strain T1S

The two enrichment cultures of ammonia oxidizing archaea from skin (isolated from a human healthy child) belong to the family of Nitrososphaeraceae of the phylum Thermoproteota (formerly Thaumarchaeota) of the domain Archaea as determined by 16S rRNA sequencing. Their morphological appearance is similar to described strains of the genus Nitrosocosmicus (like Ca. Nitrosocosmicus franklandianus, Ca. N. arcticus and Ca. N. oleophilus). The cells have a diameter of approx. 1 μm, and grow mostly in irregular shaped aggregates of 5 to 25 cells. The cell wall is visible as bright ring in phase contrast light microscopy using a 100× objective (Nikon Eclipse Ni-U). Harsh methods (like beat beating) are required to break open the rigid cell wall. The partial sequence of the amoA gene in Ca. Nitrosocosmicus ssp. R2S is 2.21% different (=13 different nt) from its closest described relative Ca. Nitrosocosmicus oleophilus strain MY3 and the sequence of Ca. Nitrosocosmicus ssp. T1S is 4.25% different (=25 different nt) from that of its closest relative Ca. Nitrosocosmicus oleophilus.

Skin X2B is 5.78% different (=34 different nt) from its closest described relative Ca. Nitrosocosmicus franklandianus and the sequence of Ca. Nitrosocosmicus ssp. Z3A is 5.1% different (=30 different nt) from that of its closest relative Ca. Nitrosocosmicus hydrocola.

The four skin strains differ between 5.1% and 10.88% from each other in their partial gene sequences (i.e., 30 to 64 different nt).

Active growth of enrichment cultures is monitored by measuring the consumption of ammonia (salicylate method with sodium salicylate, sodium nitroprusside dihydrate, dichloroisocyanuric acid sodium salt hydrate) and the production of nitrite (Griess reagent system, Promega) in regular intervals (1 day to 1 week). R2S and T1S consume approx. 0.5 mM ammmonia within 25 and 18 days, respectively. Growth rate increases with increase of bacterial cells in the cultures.

### Enrichment and Cultivation Procedure:

Donors carrying relative higher amounts of AOA on skin as compared to other test persons, were identified by performing qualitative PCR of DNA from skin swab samples with a 16S rRNA-specific primer pair (Ochsenreiter et al. 2003, Diversity and abundance of Crenarchaeota in terrestrial habitats studied by 16S RNA surveys and real time PCR. Ochsenreiter T, Selezi D, Quaiser A, Bonch-Osmolovskaya L, Schleper C. Environ Microbiol. 2003 September; 5 (9): 787-97. doi: 10.1046/j.1462-2920.2003.00476.x.PMID: 12919414). Swabs were used premoistened to sample body parts for gaining cells for cultivation. The stick of the swab was cut, and the head of the swab left in the culture tube as inoculum. The cells were grown in 20 mL FWM, (Fresh Water Medium: 1 g NaCl, 0.4 g MgCl2 6H2O, 0.1 g CaCl2 2H2O, 0.2 g KH2PO4, 0.5 g KCl per liter, plus 1 mL Modified Trace Elements (8 mL 12.5M HCl, 30 mg H3BO3, 100 mg MnCl2 4 H2O, 190 mg CoCl2 6 H2O, 24 mg NiCl2 6H2O, 2 mg CuCl2 2H2O, 144 mg ZnSO4 7H2O, 36 mg Na2MoO4 2H2O, 987 ml distilled H2O), 1 mL FeNaEDTA solution (2753 mg FeNaEDTA, filled up to 1000 mL with MilliQ water) and supplemented with 0.5 mM ammonia in 30 ml flasks. Cultures were incubated for several weeks at 28 to 30° C. without shaking until nitrite production was first observed. Relative enrichments of the AOA over contaminating bacteria were achieved by addition of antibiotics (kanamycin, carbenicillin, chloramphenicol, streptomycin, novobiocin) during passages of the culture. Growth was generally followed by measuring nitrite production and ammonia consumption from liquid samples as in Tourna et al. 2011. The abundance of the cells in culture was followed by phase contrast microscopy.

### Example 2: sAOA Isolates T1S and R2S

The specific sAOA preparation T1S is an axenic culture, meaning that all other microorganisms in the original enrichment were eliminated by a combined usage of antibiotics and filtration, as described before.

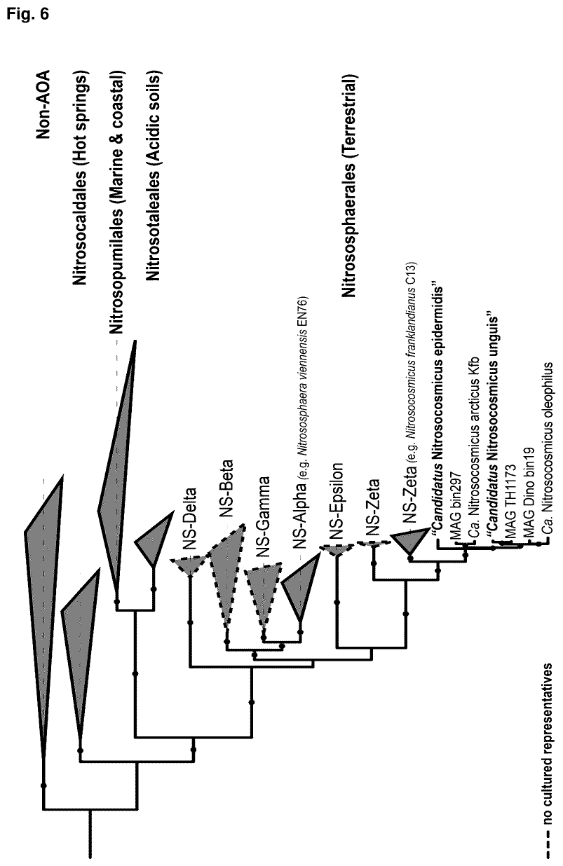

Genomes of strains T1S, R2S were resequenced with Oxford Nanopore technology generating a full closed genome. This enabled full phylogenetic investigations of these strains and their designation as two distinct species: Candidatus Nitrosocosmicus epidermidis T1S and Candidatus Nitrosocosmicus unguis R2S (FIG. 6). The phylogenomic investigations revealed the taxonomic placement of the two skin enrichments, which cluster within the genus Nitrosocosmicus. Based on average nucleotide identity values and their taxonomic placement, the two enriched AOA were identified as two distinct species, Ca. Nitrosocosmicus unguis strain R (strain R2S) and Ca. Nitrosocosmicus epidermidis strain T (strain T1S).

The novel species Ca. Nitrosocosmicus epidermidis is the first characterized archaeal isolate from human skin, and Candidatus statuses for the thaumarchaeal strains cultivated here, are proposed with the following taxonomic assignment:
Class Nitrososphaeria
Order Nitrososphaerales
Family Nitrososphaeraceae
(a) Candidatus Nitrosocosmicus epidermidis sp. nov. strain T.
(b) Candidatus Nitrosocosmicus unguis sp. nov. Strain R.

Etymology: L. adj. nitrosus, “full of natron,” here intended to mean nitrous (nitrite producer); L. masc. n. cosmicus, cosmopolitan; (a) L. fem. gen. epidermidis “on top of skin”, describing the origin of the sample from the human skin. (b) L. masc. gen. unguis “fingernail”, describing the origin of the sample from free edges of fingernails.

Source: human skin or free edges of fingernails of a European child (male, 10 years old).

Description: facultative ammonia-oxidizing archaea of the phylum Thermoproteota (GTDB), able to utilize ammonium as substrate for ammonia oxidation at temperatures between 28 and 32° C. (a) or 28° C. (b), at pH ranging from 7 (a) or 7 to 8.5 (b); spherically shaped with a diameter of 1-2 μm.

Differentiation relative to closest relative: Based on average nucleotide identity (ANI) between all Ca. Nitrosocosmicus strains (<94%) and extensive differences in protein-coding gene complement, a separate species name for species epidermidis and unguis is proposed.

Investigation of the genomes of the sAOA isolates T1S and R2S revealed specific adaptations that hypothetically might facilitate colonization and persistence of the human skin biotope (adhesins and serralysin-like metalloproteases cysteine-rich secretory protein family proteins, triacylglycerol esterases/lipases). In addition, N. epidermidis encodes a putative α-arrestin-like protein, which might enable communication with the skin epithelial cells.

Cultivation of sAOA from human skin microbiome was performed as follows.

Methods: A combination of cultivation-dependent and -independent methods was applied to characterize, describe and enlighten the role of AOA on healthy human skin. Targeted enrichments were used to grow AOAs from human skin samples and they were characterized by phase and fluorescence microscopy. Targeted qualitative (16S rRNA and amoA gene amplicon profilings), quantitative (16S rRNA and amoA gene based qPCR) and untargeted (shotgun and Nanopore metagenomic sequencing) molecular approaches were used in gene and genome-centric manners. 47 subjects were recruited in two cross-sectional and two longitudinal cohorts with an age range of 21 till 88 years. Captured demographic characteristics and recorded lifestyle behavior and sampled eight distinct body sites distinguished by measured skin physiological parameters like skin health, pH, skin fat, moisture and temperature. In total, more than 1,780 samples were processed to describe the role of AOA on human skin.

Results: After an extensive period of analyses and optimizations, four stable enrichment cultures of the new species Ca. Nitrosocosmicus epidermidis (T1S) could be grown in the laboratory that reached nitrite concentrations of around 450 UM every two-four weeks with 10% inoculation volume. According to microscopy the coccoid shaped cells of 1-2 μm in diameter seem to form aggregates of putative extra polymeric substances and autofluorescence of the F420 conserved cofactor was visible. Metagenomic sequencing (shotgun and Nanopore) enabled the reconstruction of high quality (>99% completeness) metagenome assembled genomes (MAGs) and retrieval of 16S rRNA marker genes most closely related toN. arcticus andN. oleophilus. MAGs revealed a high proportion of unique genomic features (>560) including an array of extracellular peptidases, glycosylases, sodium/solute symporter superfamily genes and cell surface related proteins which could enable the adaptation to the skin environment. Furthermore, a gene of the glucose-methanol-choline oxidoreductase (GMC) family was found that could oxidize typical skin compounds like choline, cellobiose or other alcohols. A comparative marker gene (16S rRNA and amoA genes) analysis revealed a similarity of 99.66% over a length of approx. 300 bp between MAGs and AOA signatures obtained by amplicon sequencing of diverse cohorts. When a universal primer construct was used 9.45+/−5.54% of all processed samples were positive for archaea and an archaea specific primer setup could even detect archaea in 69.98+/−18.05% of all samples. Elderly subjects showed a higher diversity of archaea on their skin (up to H′ ~3) and quantitative PCR showed highest proportions of AOA on samples from the head, which was particularly characterized by lower pH, lower transepidermal water loss and a higher sebum content. A specific co-occurrence between Nitrosocosmicus and other skin bacteria could be identified, indicating a stable longitudinal integration of this archaeal genus into skin microbial communities of healthy subjects and unsuspicious on skin sites affected by psoriasis.

Conclusion: Ca. Nitrosocosmicus epidermidis (T1S) is wide-spread and stable associated with the healthy human skin microbiome especially on the head region, while Nitrososphaera and other archaeal species are likely only transient archaeal signatures in this habitat.

### REFERENCES

1. Byrd A L, Belkaid Y, Segre J A. The human skin microbiome. Nat Rev Microbiol 2018; 16:143-155.
2. Chen Y E, Fischbach M A, Belkaid Y. Skin microbiota-host interactions. 2018; 553:427-436.
3. Naik S, Bouladoux N, Wilhelm C, Molloy M J, Salcedo R, Spencer S, et al. Compartmentalized Control of Skin. Science 2012; 337:1115-1120.
4. Belkaid Y, Tamoutounour S. The influence of skin microorganisms on cutaneous immunity. Nat Rev Immunol 2016; 16:353-366.
5. Altunbulakli C, Reiger M, Neumann A U, Garzorz-Stark N, Fleming M, Huelpuesch C, et al. Relations between epidermal barrier dysregulation and Staphylococcus species-dominated microbiome dysbiosis in patients with atopic dermatitis. J Allergy Clin Immunol 2018; 142:1643-1647.e12.
6. Zeeuwen PLJM, Kleerebezem M, Timmerman H M, Schalkwijk J. Microbiome and skin diseases. Curr Opin Allergy Clin Immunol 2013; 13:514-520.
7. Alekseyenko A V, Perez-Perez G I, De Souza A, Strober B, Gao Z, Bihan M, et al. Community differentiation of the cutaneous microbiota in psoriasis. Microbiome 2013; 1:1-17.
8. Chng K R, Tay A S L, Li C, Ng A H Q, Wang J, Suri B K, et al. Whole metagenome profiling reveals skin microbiome-dependent susceptibility to atopic dermatitis flare. Nat Microbiol 2016; 1:1-10.
9. Alani J I, Davis M D P, Yiannias J A. Allergy to Cosmetics. Dermatitis 2013; 24:283-290.
10. Holland K T, Bojar R A. Cosmetics: What is their influence on the skin microflora? Am J Clin Dermatol 2002; 3:445-449.
11. Lunjani N, Hlela C, O'Mahony L. Microbiome and skin biology. Curr Opin Allergy Clin Immunol 2019; 19:328-333.
12. Paetzold B, Willis J R, Pereira De Lima J, Knödlseder N, Brüggemann H, Quist S R, et al. Skin microbiome modulation induced by probiotic solutions. Microbiome 2019; 7:1-9.
13. Probst A J, Auerbach A K, Moissl-Eichinger C. Archaea on human skin. PLOS One 2013; 8: e65388.
14. Borrel G, Brugere J-F, Gribaldo S, Schmitz R A, Moissl-Eichinger C. The host-associated archaeome. Nat Rev Microbiol 2020.
15. Koskinen K, Pausan M R, Perras A K, Beck M, Bang C, Mora M, et al. First insights into the diverse human archaeome: Specific detection of Archaea in the gastrointestinal tract, lung, and nose and on skin. MBio 2017; 8:1-17.
16. Pausan M R, Csorba C, Singer G, Till H, Schopf V, Santigli E, et al. Exploring the Archaeome: Detection of Archaeal Signatures in the Human Body. Front Microbiol 2019; 10:1-13.
17. Ross A A, Müller K M, Scott Weese J, Neufeld J D. Comprehensive skin microbiome analysis reveals the uniqueness of human skin and evidence for phylosymbiosis within the class Mammalia. Proc Natl Acad Sci USA 2018; 115: E5786-E5795.
18. Oh J, Byrd A L, Deming C, Conlan S, Kong H H, Segre J A, et al. Biogeography and individuality shape function in the human skin metagenome. Nature 2014; 514:59-64.
19. Moissl-Eichinger C, Probst A J, Birarda G, Auerbach A, Koskinen K, Wolf P, et al. Human age and skin physiology shape diversity and abundance of Archaea on skin. Sci Rep 2017; 7:4039.
20. Stahl D A, de la Torre J R. Physiology and diversity of ammonia-oxidizing archaea. Annu Rev Microbiol 2012; 66:83-101.
21. Schleper C, Nicol G W. Ammonia-oxidising archaea—physiology, ecology and evolution. Adv Microb Physiol 2010; 57:1-41.
22. Kozlowski J A, Stieglmeier M, Schleper C, Klotz M G, Stein L Y. Pathways and key intermediates required for obligate aerobic ammonia-dependent chemolithotrophy in bacteria and Thaumarchaeota. ISME J 2016; 10:1836-1845.
23. Rapozzi V, Della Pietra E, Bonavida B. Dual roles of nitric oxide in the regulation of tumor cell response and resistance to photodynamic therapy. Redox Biol 2015; 6:311-317.
24. Bogdan C. Nitric oxide synthase in innate and adaptive immunity: An update. Trends Immunol 2015; 36:161-178.
25. Cals-Grierson M M, Ormerod A D. Nitric oxide function in the skin. Nitric Oxide-Biol Chem 2004; 10:179-193.
26. Bogdan C. Nitric oxide and the immune response. Nat Immunol 2001; 2:907-916.
27. Brugere J F, Borrel G, Gaci N, Tottey W, O'Toole P W, Malpuech-Brugere C. Archaebiotics: Proposed therapeutic use of archaea to prevent trimethylaminuria and cardiovascular disease. Gut Microbes 2013; 5.
28. Nodake Y, Matsumoto S, Miura R, Honda H, Ishibashi G, Matsumoto S, et al. Pilot study on novel skin care method by augmentation with Staphylococcus epidermidis, an autologous skin microbe-A blinded randomized clinical trial. J Dermatol Sci 2015; 79:119-126.
29. LeeN. Nitrosomonas Eutropha in the Treatment of Keratosis Pilaris: A Randomized Clinical Trial. J Drugs Dermatology 2018.
30. Nakatsuji T, Chen T H, Narala S, Chun K A, Two A M, Yun T, et al. Antimicrobials from human skin commensal bacteria protect against Staphylococcus aureus and are deficient in atopic dermatitis. Sci Transl Med 2017; 9: eaah4680.
31. Abby S S, Melcher M, Kerou M, Krupovic M, Stieglmeier M, Rossel C, et al. Candidatus Nitrosocaldus cavascurensis, an Ammonia Oxidizing, Extremely Thermophilic Archaeon with a Highly Mobile Genome. Front Microbiol 2018; 9:28.
32. Abby S S, Kerou M, Schleper C. Ancestral reconstructions decipher major adaptations of ammonia oxidizing archaea upon radiation into moderate terrestrial and marine environments, bioRxiv 2020; 2020.06.28.176255.
33. Valentine D L. Adaptations to energy stress dictate the ecology and evolution of the Archaea. Nat Rev Microbiol 2007; 5:316-323.
34. Martens-Habbena W, Berube P M, Urakawa H, de la Torre J R, Stahl D A. Ammonia oxidation kinetics determine niche separation of nitrifying Archaea and Bacteria. Nature 2009; 461:976-979.
35. Kits K D, Sedlacek C J, Lebedeva E V., Han P, Bulaev A, Pjevac P, et al. Kinetic analysis
36. of a complete nitrifier reveals an oligotrophic lifestyle. Nature 2017; 549: pages 269-272. Bang C, Schmitz R A. Archaea associated with human surfaces: Not to be underestimated. FEMS Microbiol Rev 2015; 39:631-648.
37. Oliver S, Pham T T P, Li Y, Xu F J, Boyer C. More than skin deep: Using polymers to facilitate topical delivery of nitric oxide. Biomater Sci 2021; 9:391-405.
38. Maura D, Elmekki N, Goddard C A. The ammonia oxidizing bacterium Nitrosomonas eutropha blocks T helper 2 cell polarization via the anti-inflammatory cytokine IL-10. Sci Rep 2021; 11:1-10.
39. Zipperer A, Konnerth M C, Laux C, Berscheid A, Janek D, Weidenmaier C, et al. Human commensals producing a novel antibiotic impair pathogen colonization. Nature 2016; 535:511-516.
40. Doxey A C, Kurtz D A, Lynch M D, Sauder L A, Neufeld J D. Aquatic metagenomes implicate Thaumarchaeota in global cobalamin production. ISME J 2015; 9:461-71.
41. Lim S K, Kwon M S, Lee J, Oh Y J, Jang J Y, Lee J H, et al. Weissella cibaria WIKIM28 ameliorates atopic dermatitis-like skin lesions by inducing tolerogenic dendritic cells and regulatory T cells in BALB/c mice. Sci Rep 2017; 7:1-9.
42. https://www.aobiome.com.
43. Gryllos I, Ghosh S, Vajrala N, Whitlock D, Sayavedra-Soto L, Jamas S. Ammonia-oxidizing bacteria accelerate wound closure in diabetic mice. Nitric Oxide 2014; 42:111-112.
44. Caporaso J G, Lauber C L, Costello E K, Berg-Lyons D, Gonzalez A, Stombaugh J, et al. Moving pictures of the human microbiome. Genome Biol 2011; 12: R50.
45. Zhang L-M, Hu H-W, Shen J-P, He J-Z. Ammonia-oxidizing archaea have more important role than ammonia-oxidizing bacteria in ammonia oxidation of strongly acidic soils. ISME J 2012; 6:1032-1045.
46. Gubry-Rangin C, Novotnik B, Mandič-Mulec I, Nicol G W, Prosser J I. Temperature responses of soil ammonia-oxidising archaea depend on pH. Soil Biol Biochem 2017; 106:61-68.
47. Alves R J E, Wanek W, Zappe A, Richter A, Svenning M M, Schleper C, et al. Nitrification rates in Arctic soils are associated with functionally distinct populations of ammonia-oxidizing archaea. 2013; 7:1620-1631.
48. Taylor A E, Giguere A T, Zoebelein C M, Myrold D D, Bottomley P J. Modeling of soil nitrification responses to temperature reveals thermodynamic differences between ammonia-oxidizing activity of archaea and bacteria. ISME J 2017; 11:896-908.
49. Konneke M, Schubert D M, Brown P C, Hugler M, Standfest S, Schwander T, et al. Ammonia-oxidizing archaea use the most energy-efficient aerobic pathway for CO2 fixation. Proc Natl Acad Sci USA 2014; 111:8239-8244.
50. Prosser J I, Nicol G W. Archaeal and bacterial ammonia-oxidisers in soil: the quest for niche specialisation and differentiation. Trends Microbiol 2012; 20:523-531.
51. Leung M H Y, Wilkins D, Lee P K H. Insights into the pan-microbiome: skin microbial communities of Chinese individuals differ from other racial groups. Sci Rep 2015; 5:11845.
52. Sedlacek C J. It Takes a Village: Discovering and Isolating the Nitrifiers. Front Microbiol 2020; 11:1900.
53. Pfeifer K, Ergal i, Koller M, Basen M, Schuster B, Rittmann SKMR. Archaea Biotechnology. Biotechnol Adv 2021; 47.
54. Stieglmeier M, Klingl A, Alves R J, Rittmann S K, Melcher M, Leisch N, et al. Nitrososphaera viennensis gen. nov., sp. nov., an aerobic and mesophilic, ammonia-oxidizing archaeon from soil and a member of the archaeal phylum Thaumarchaeota. Int J Syst Evol Microbiol 2014; 64:2738-2752.
55. Liu B, Zheng D, Jin Q, Chen L, Yang J. VFDB 2019: a comparative pathogenomic platform with an interactive web interface. Nucleic Acids Res 2019; 47: D687-D692.
56. Hofmann E, Fink J, Eberl A, Prugger E-M, Kolb D, Luze H, et al. A novel human ex vivo skin model to study early local responses to burn injuries. Sci Rep 2021; 11:364.
57. Hyatt, D. et al. Prodigal: prokaryotic gene recognition and translation initiation site identification. BMC Bioinformatics 11, 119 (2010).
58. Emms, D. M. & Kelly, S. OrthoFinder: Phylogenetic orthology inference for comparative genomics. Genome Biol. 20, 1-14 (2019).
59. Katoh, K. & Standley, D. M. MAFFT multiple sequence alignment software version 7: Improvements in performance and usability. Mol. Biol. Evol. 30, 772-780 (2013).
60. Minh, B. Q. et al. IQ-TREE 2: New Models and Efficient Methods for Phylogenetic Inference in the Genomic Era. Mol. Biol. Evol. 37, 1530-1534 (2020).
61. Rodriguez-R, L. M. & Konstantinidis, K. T. The enveomics collection: a toolbox for specialized analyses of microbial genomes and metagenomes. PeerJ Prepr. 4, e1900v1 (2016).
62. Rinke, C., Chuvochina, M., Mussig, A. J. et al. A standardized archaeal taxonomy for the Genome Taxonomy Database. Nat Microbiol 6, 946-959 (2021)
63. Pierre-Alain Chaumeil, Aaron J Mussig, Philip Hugenholtz, Donovan H Parks, GTDB-Tk: a toolkit to classify genomes with the Genome Taxonomy Database, Bioinformatics, Volum
64. Graeme W. Nicol et al., Microbiology Resource Announcements, 2019, vol. 8, no. 40, pp. e00435-19

1. A preparation comprising skin-specific ammonia-oxidizing archaea (SAOA) of the phylum Thermoproteota and genus Nitrosocosmicus, which comprise at least one of the ssp strains R2S, T1S, X2B, or Z3A.

2. The preparation of claim 1, wherein at least one of said sAOA comprises an amoA gene comprising a sequence that is distinct from amoA gene sequences of any other Nitrosocosmicus strains.

3. The preparation of claim 1, wherein
a) the amoA gene sequence of ssp strain R2S comprises or consists of SEQ ID NO: 1;
b) the amoA gene sequence of ssp strain T1S comprises or consists of SEQ ID NO: 2;
c) the amoA gene sequence of ssp strain X2B comprises or consists of SEQ ID NO: 3;
d) the amoA gene sequence of ssp strain Z3A comprises or consists of SEQ ID NO: 4.

4. The preparation of claim 1, which comprises further ammonia-oxidizing archaea (AOA) originating from environmental sources preferably human skin, soil or marine sources, preferably of the family of Nitrososphaeraceae, preferably selected from one or more of:
a) the genus Nitrososphaera, preferably Nitrososphaera viennensis, preferably Nitrososphaera viennensis EN76, Nitrososphaera viennensis EN123, or Nitrososphaera viennensis KFJ;
b) the genus Nitrosocosmicus, preferably Nitrosocosmicus oleophilus, Nitrosocosmicus arcticus, Nitrosocosmicus hydrocola or Nitrosocosmicus franklandianus, preferably Ca. Nitrosocosmicus oleophilus strain MY3, Ca. Nitrosocosmicus arcticus, Ca. Nitrosocosmicus sp. (Mir11), Nitrosocosmicus franklandianus C13, or Nitrosocosmicus hydrocola G61.

5. The preparation of claim 1, wherein the SAOA are obtained by a method of growing the sAOA in a cell culture and isolating the sAOA from the cell culture, optionally wherein the sAOA are inactivated and/or fractionated.

6. The preparation of claim 1, wherein the cell culture is an sAOA monoculture or co-culture with other AOA.

7. A composition comprising the preparation of claim 1 and a cosmetic carrier, preferably in the form of a cream, emulsion, gel, ointment, lotion, spray, aerosol, foam, solution, dry powder, bath salt, bath ball, tablet, shampoo, conditioner, deodorant, soap, skin gel, lip balm, facial mask, or makeup.

8. A cosmetic composition comprising the composition of claim 7.

9. Use of the composition of claim 8, for topical or mucosal application to a human body.

10. Use of the composition of claim 9 for applying to skin, hair or mucosa.

11. The use of claim 9, wherein said application is to the surface of the skin, a wound, and/or mucosal cells or tissues, preferably alveolar, buccal, dermal, lingual, masticatory, or nasal mucosa.

12. The use of claim 9, for any one or more of the following: regulating the microbiome, moisturizing, purifying, achieving anti-stinging, soothing, anti-aging, or improving barrier function of skin, hair or mucosa.

13. A pharmaceutical preparation comprising the preparation of claim 1, and a pharmaceutically acceptable carrier, preferably formulated in the form of a cream, emulsion, gel, ointment, lotion, spray, aerosol, pellet or dry powder.

14. The preparation of claim 1, for use as a medicament.

15. The preparation of claim 13, for use in the medical treatment of a human subject wherein the preparation is administered to body surfaces, preferably skin, hair or mucosa, in an effective amount to modulate the subject's microbiome.

16. A preparation of claim 1 for use as a probiotic in the treatment of a subject for accelerating wound healing, or preventing or treating skin diseases.

17. The preparation for use according to claim 16, wherein the subject is suffering from a dermatologic disease or disorder, preferably acne, psoriasis, atopic dermatitis, eczema, allergic inflammation, skin hypersensitivity, UV-induced skin damage, skin inflammation or skin redness.

18. A method of producing a preparation of claim 1, comprising:
a) culturing the sAOA in a cell culture,
b) separating the sAOA from the cell culture, and
c) formulating the sAOA preparation;
optionally wherein the sAOA are inactivated and/or fractionated.
